(19) United States
(12) Patent Application Publication
(10) Pub. No.: US 2022/0218181 A1
(43) Pub. Date: Jul. 14, 2022

(54) PROCESSING APPARATUS, PROCESSING METHOD, AND COMPUTER READABLE RECORDING MEDIUM

(71) Applicant: OLYMPUS CORPORATION, Tokyo (JP)

(72) Inventor: Naoto KOIDE, Tokyo (JP)

(73) Assignee: OLYMPUS CORPORATION, Tokyo (JP)

(21) Appl. No.: 17/708,352

(22) Filed: Mar. 30, 2022

Related U.S. Application Data
(63) Continuation-in-part of application No. PCT/JP2019/038842, filed on Oct. 2, 2019.

(51) Int. Cl. A61B 1/00; A61B 1/04

(52) U.S. Cl. CPC A61B 1/00016; A61B 1/273; A61B 1/00009; A61B 1/041

(57) ABSTRACT

A processing apparatus includes: a processor configured to: acquire, for each of receiving antennas receiving wireless signals that are transmitted from a medical apparatus configured to capture an image of an inside of a subject, a plurality of pieces of line data that constitute first image data from the wireless signals, count numbers related to acquisition of synchronous signals included in the plurality of pieces of line data, and collect a plurality of pieces of line data that constitute second image data from the plurality of pieces of line data that are acquired by the receiving antennas and that constitute a plurality of pieces of the first image data, based on the numbers related to acquisition.

### CROSS-REFERENCE TO RELATED APPLICATION

This application is a continuation-in-part of International Application No. PCT/JP2019/038842, filed on Oct. 2, 2019, the entire contents of which are incorporated herein by reference.

### BACKGROUND

### 1. Technical Field

The present disclosure relates to a processing apparatus, a processing method, and a computer readable recording medium.

### 2. Related Art

In the relater art, an endoscope has been in widespread use as a medical observation apparatus that is introduced into an inside of a body of a subject, such as a patient, and observes the inside of the body of the subject. Further, in recent years, a capsule endoscope as a swallow-type image acquisition apparatus that includes, inside a capsule-shaped casing, an imaging apparatus, a communication apparatus that wirelessly transmits an image signal captured by the imaging apparatus to outside the body, and the like has been developed (see, for example, Japanese Laid-open Patent Publication No. 2007-75161). The capsule endoscope, after being swallowed through a mouth of a patient for observation of an inside of the body of the subject and until being naturally excreted from the subject, moves inside of organs, such as an esophagus, a stomach, and a small intestine, in accordance with peristaltic movement of the organs and sequentially captures images.

While the capsule endoscope moves inside the body of the subject, image data captured by the capsule endoscope is sequentially transmitted to the outside of the body by wireless communication, and stored in a memory provided inside or outside of a receiving apparatus located outside the body via a receiving antenna or displayed as an image on a display that is arranged on the receiving apparatus. A doctor or a nurse is able to load the image data stored in the memory onto an information processing apparatus via a cradle to which the receiving apparatus is inserted and make a diagnosis on the basis of an image displayed on the information processing apparatus.

A plurality of receiving antennas, each of which receives image data transmitted by the capsule endoscope, are attached to the subject. The information processing apparatus selects a receiving antenna with the highest reception strength and acquires image data that has been received by the selected receiving antenna.

### SUMMARY

In some embodiments, a processing apparatus includes: a processor configured to: acquire, for each of receiving antennas receiving wireless signals that are transmitted from a medical apparatus configured to capture an image of an inside of a subject, a plurality of pieces of line data that constitute first image data from the wireless signals, count numbers related to acquisition of synchronous signals included in the plurality of pieces of line data, and collect a plurality of pieces of line data that constitute second image data from the plurality of pieces of line data that are acquired by the receiving antennas and that constitute a plurality of pieces of the first image data, based on the numbers related to acquisition.

In some embodiments, provided is a processing method implemented by a processor. The processing method includes: acquiring, for each of receiving antennas receiving wireless signals that are transmitted from a medical apparatus configured to capture an image of an inside of a subject, a plurality of pieces of line data that constitute first image data from the wireless signals; counting numbers related to acquisition of synchronous signals included in the plurality of pieces of line data; and collecting a plurality of pieces of line data that constitute second image data from the plurality of pieces of line data that are acquired by the receiving antennas and that constitute a plurality of pieces of the first image data, based on the numbers related to acquisition.

In some embodiments, provided is a non-transitory computer readable recording medium having an executable program recorded thereon. The program instructs a processor to execute: acquiring, for each of receiving antennas receiving wireless signals that are transmitted from a medical apparatus configured to capture an image of an inside of a subject, a plurality of pieces of line data that constitute first image data from the wireless signals; counting numbers related to acquisition of synchronous signals included in the plurality of pieces of line data; and collecting a plurality of pieces of line data that constitute second image data from the plurality of pieces of line data that are acquired by the receiving antennas and that constitute a plurality of pieces of the first image data, based on the numbers related to acquisition.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system according to a first embodiment of the present disclosure;

FIG. 2 is a block diagram illustrating a schematic configuration of the capsule endoscope system according to the first embodiment of the present disclosure;

FIG. 3 is a block diagram illustrating a configuration of a receiving antenna in the capsule endoscope system according to the first embodiment of the present disclosure;

FIG. 4 is a diagram for explaining a configuration of data stored in a storage unit according to the first embodiment of the present disclosure;

FIG. 5 is a flowchart illustrating an image data acquisition process performed by the capsule endoscope system according to the first embodiment of the present disclosure;

FIG. 6 is a flowchart illustrating an image data acquisition process performed by a capsule endoscope system according to a second embodiment of the present disclosure;

FIG. 7 is a flowchart illustrating an image data acquisition process performed by a capsule endoscope system according to a third embodiment of the present disclosure;

FIG. 8 is a flowchart illustrating an image data acquisition process performed by a capsule endoscope system according to a fourth embodiment of the present disclosure;

FIG. 9 is a diagram for explaining a configuration of line data used in the fourth embodiment of the present disclosure;

FIG. 10 is a schematic diagram illustrating a schematic configuration of an endoscope system according to a fifth embodiment of the present disclosure; and

FIG. 11 is a block diagram illustrating the schematic configuration of the endoscope system according to the fifth embodiment of the present disclosure.

### DETAILED DESCRIPTION

A capsule endoscope system using a medical capsule endoscope will be described below as embodiments of the present disclosure. In description of the drawings, the same components are denoted by the same reference symbols. Further, it is necessary to note that the drawings are schematic, and a relation between a thickness and a width of each of the components, ratios among the components, and the like are different from actual ones.

### First Embodiment

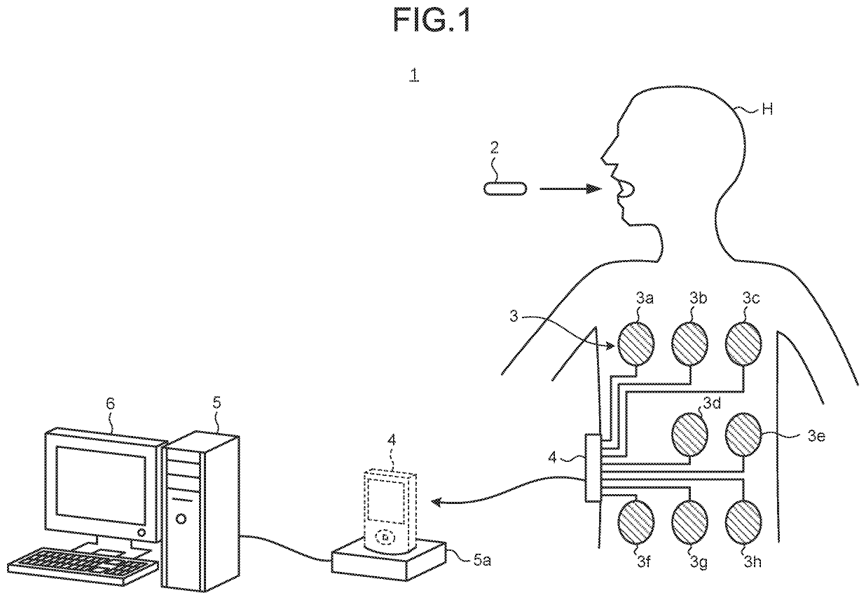

FIG. 1 is a schematic diagram illustrating a schematic configuration of a capsule endoscope system according to a first embodiment of the present disclosure. A capsule endoscope system 1 illustrated in FIG. 1 includes a capsule endoscope 2 that is introduced into a subject H, that generates image data by capturing an image of an inside of the subject H, that superimposes the image data on a wireless signal, and that transmits the wireless signal by radio waves, a receiving apparatus 4 that receives the wireless signal transmitted from the capsule endoscope 2 via a receiving antenna unit 3 including a plurality of receiving antennas (receiving antennas 3a to 3h) attached to the subject H, and a processing apparatus 5 that loads the image signal captured by the capsule endoscope 2 from the receiving apparatus 4 via a cradle 5a, that performs processing on the image data, and that generates image data representing an image of the inside of the subject H. The image data generated by the processing apparatus 5 is displayed and output by a display apparatus 6, for example. A receiving system is configured with the plurality of receiving antennas and the receiving apparatus 4.

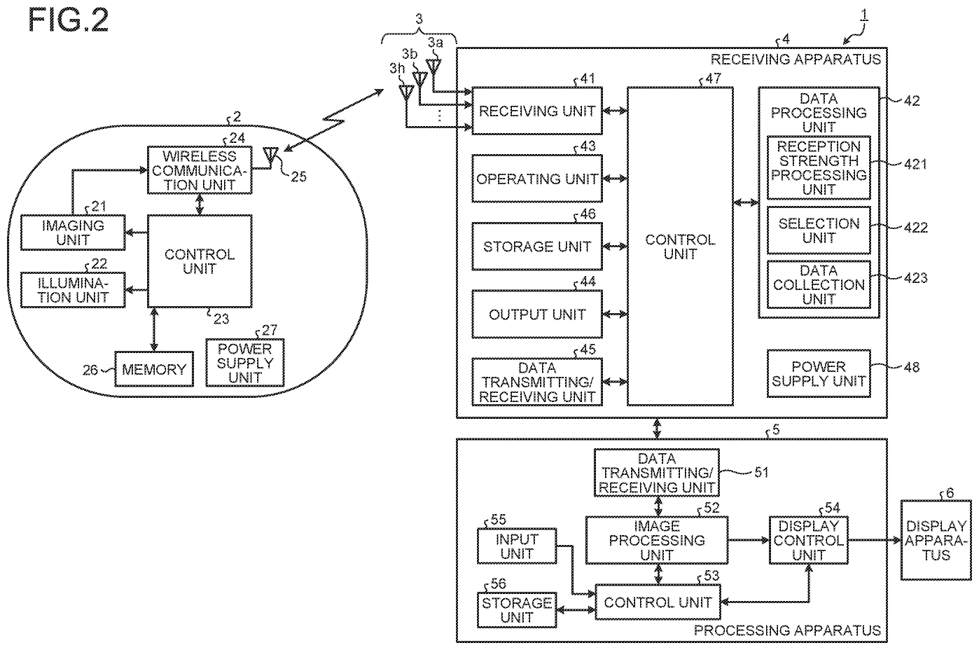

FIG. 2 is a block diagram illustrating a schematic configuration of the capsule endoscope system according to the first embodiment of the present disclosure. The capsule endoscope 2 includes an imaging unit 21, an illumination unit 22, a control unit 23, a wireless communication unit 24, a memory 26, and a power supply unit 27. The capsule endoscope 2 is an apparatus in which the components as described above are incorporated in a capsule-shaped casing with a size that can be swallowed by the subject H.

The imaging unit 21 includes an image sensor that generates, from an optical image formed on a light receiving surface, image data in which the inside of the subject H is captured and outputs the image data, and an optical system, such as an objective lens, that is arranged at a side of the light receiving surface of the image sensor, for example. The image sensor includes a plurality of pixels that are arranged in a matrix manner and that receive light from the subject H, and generates image data by performing photoelectric conversion on light received by the pixels. The imaging unit 21 reads pixel values of the plurality of pixels, which are arranged in a matrix manner, for each of horizontal lines, and generates image data including a plurality of pieces of line data to each of which a synchronous signal is assigned for each of the horizontal lines. The imaging unit 21 is configured with a charge coupled device (CCD) image sensor or a complementary metal oxide semiconductor (CMOS) image sensor.

The illumination unit 22 is configured with a white light emitting diode (LED) or the like that generates white light as illumination light. Meanwhile, it may be possible to adopt a configuration that generates white light by combining light from a plurality of LEDs or laser light sources with different emission wavelength bands or a configuration using a xenon lamp, a halogen lamp, or the like, instead of the white LED.

The control unit 23 controls an operation process of each of the components of the capsule endoscope 2. For example, when the imaging unit 21 performs an imaging process, the control unit 23 causes the image sensor to perform an exposure process and a read process and causes the illumination unit 22 to emit illumination light in accordance with an exposure timing of the imaging unit 21. The control unit 23 is configured with a general-purpose processor, such as a central processing unit (CPU), or a dedicated processor, such as various arithmetic circuits including an application specific integrated circuit (ASIC) or the like, that implements specific functions.

The wireless communication unit 24 performs a modulation process on the image data that is output from the imaging unit 21, and transmits the image data to outside. The wireless communication unit 24 performs analog to digital (A/D) conversion and predetermined signal processing on the image data that is output from the imaging unit 21, acquires image data in a digital format, superimposes the image data together with related information on a wireless signal, and transmits the wireless signal from an antenna 25. The related information includes identification information (for example, a serial number) on the capsule endoscope 2, which is assigned to identify the individual capsule endoscope 2, identification information (for example, a captured image number to be described later) on image data to be transmitted, and the like. Meanwhile, the wireless communication unit 24 may be configured to receive a control signal transmitted from the receiving apparatus 4 via the antenna 25.

The memory 26 stores therein an execution program and a control program that are used by the control unit 23 to execute various kinds of operation and a parameter, such as a threshold. The memory 26 is configured with a volatile memory, a non-volatile memory, or a combination of a volatile memory and a non-volatile memory. The memory 26 is configured with a random access memory (RAM), a read only memory (ROM), or the like.

The power supply unit 27 includes a battery including a button battery or the like, a power supply circuit that supplies electric power to each of the units, and a power switch for switching between an ON state and an OFF state of the power supply unit 27, and supplies electric power to each of the units in the capsule endoscope 2 after the power switch is turned on. Meanwhile, the power switch is configured with, for example, a reed switch for which an ON state and an OFF state are switched by an external magnetic force, and is switched to the ON state by external application of a magnetic force to the capsule endoscope 2 before use of the capsule endoscope 2 (before the capsule endoscope 2 is swallowed by the subject H).

The capsule endoscope 2 as described above, after being swallowed by the subject H, moves inside a digestive tract of the subject H by peristaltic movement or the like of organs and sequentially captures images of living body sites (an esophagus, a stomach, a small intestine, a large intestine, and the like) with a predetermined cycle (for example, with a cycle of 0.5 second). Further, an image signal and related information that are acquired by the imaging operation as described above are sequentially and wirelessly transmitted to the receiving apparatus 4.

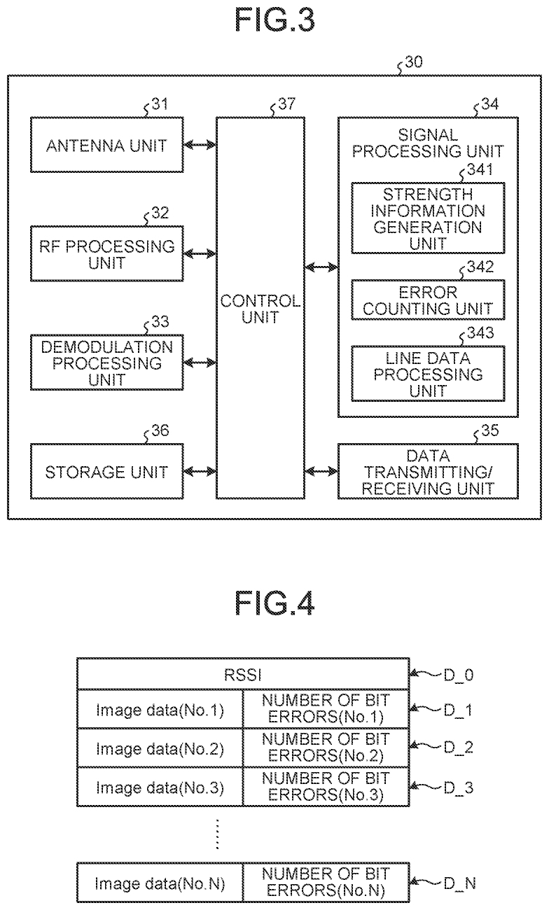

Configurations of the receiving antennas 3a to 3h will be described below with reference to FIG. 3. FIG. 3 is a block diagram illustrating a configuration of a receiving antenna in the capsule endoscope system according to the first embodiment of the present disclosure. The receiving antennas 3a to 3h have the same configurations. In FIG. 3, a configuration example of a certain receiving antenna is illustrated and is applied to the receiving antennas 3a to 3h. A receiving antenna 30 illustrated in FIG. 3 includes an antenna unit 31, a radio frequency (RF) processing unit 32, a demodulation processing unit 33, a signal processing unit 34, a data transmitting/receiving unit 35, a storage unit 36, and a control unit 37.

The antenna unit 31 receives a wireless signal from the capsule endoscope 2. The antenna unit 31 is configured with, for example, a dipole antenna, a monopole antenna, a chip antenna, or the like.

The RF processing unit 32 performs processing on an RF signal that is received by the antenna unit 31. The RF processing unit 32 extracts, from the RF signal, a signal with a frequency corresponding to data to be acquired. The RF processing unit 32 is configured with one or more of a general-purpose processor, such as a CPU, and a dedicated processor, such as various arithmetic circuits including an ASIC or the like, that implements specific functions.

The demodulation processing unit 33 demodulates the signal processed by the RF processing unit 32. The demodulation processing unit 33 performs a demodulation process on the basis of the signal with the frequency extracted from the RF signal. The demodulation processing unit 33 is configured with a general-purpose processor, such as a CPU, or a dedicated processor, such as various arithmetic circuits including an ASIC or the like, that implements specific functions.

The signal processing unit 34 performs processing on the data that has been subjected to the demodulation process. The signal processing unit 34 includes a strength information generation unit 341, an error counting unit 342, and a line signal processing unit 343. The signal processing unit 34 is configured with a general-purpose processor, such as a CPU, or a dedicated processor, such as various arithmetic circuits including an ASIC or the like, that implements specific functions. The error counting unit 342 corresponds to a counting unit.

The strength information generation unit 341 generates strength information on the RF signal that is received by the antenna unit 31. The strength information generation unit 341 measures a received signal strength indicator (RSSI) of a wireless signal that is received by each of the receiving antennas 3a to 3h. The strength information generation unit 341 outputs the measured RSSI of each of the receiving antennas 3a to 3h as the strength information.

The error counting unit 342 counts the number of bit errors of image data that is included in the signal subjected to the demodulation process, and outputs a counting result as bit error information. Specifically, the error counting unit 342 determines whether synchronous signals of a plurality of pieces of line data included in the image data are accurately received, and counts the number of bit errors in accordance with a determination result. The line data corresponds to data of a pixel sequence in a pixel array of the imaging unit 21, and a synchronous signal is added at the top or the end of the line data, for example.

The line signal processing unit 343 performs a process of associating the line data included in the image data with a frame number or a receiving antenna from the RF signal, and stores the processed line data in the storage unit 36.

The data transmitting/receiving unit 35, when being connected to the receiving apparatus 4 in a communicable manner, transmits the image data stored in the storage unit 36, information on a reception strength, or the like to the receiving apparatus 4. The data transmitting/receiving unit 35 is configured with a communication interface, such as a serial peripheral interface (SPI).

The storage unit 36 stores therein a program for causing the receiving antenna 30 to operate and implement various functions, image data received by the antenna unit 31, and the like. The storage unit 36 is configured with a RAM, a ROM, a volatile memory, or the like.

The control unit 37 controls each of the structural units of the receiving antenna 30. The control unit 37 is configured with a general-purpose processor, such as a CPU, or a dedicated processor, such as various arithmetic circuits including an ASIC or the like, that implements specific functions.

Referring back to FIG. 2, the receiving apparatus 4 includes a receiving unit 41, a data processing unit 42, an operating unit 43, an output unit 44, a data transmitting/receiving unit 45, a storage unit 46, a control unit 47, and a power supply unit 48.

The receiving unit 41 receives a wireless signal that is wirelessly transmitted by the capsule endoscope 2. Specifically, the receiving unit 41 receives image data and related information that are wirelessly transmitted by the capsule endoscope 2 via the receiving antenna unit 3.

The data processing unit 42 performs processing on the data that is received by the receiving unit 41. The data processing unit 42 includes a reception strength processing unit 421, a selection unit 422, and a data collection unit 423. The data processing unit 42 is configured with a general-purpose processor, such as a CPU, or a dedicated processor, such as various arithmetic circuits including an ASIC or the like, that implements specific functions.

The reception strength processing unit 421 acquires the RSSI of each of the receiving antennas and determines a magnitude relationship.

The selection unit 422 selects a receiving antenna that meets a predetermined condition on the basis of the magnitude relationship determined by the reception strength processing unit 421. For example, if a receiving antenna with the largest RSSI is to be selected, the selection unit 422 selects a receiving antenna with the largest RSSI on the basis of the magnitude relationship as described above. Further, the selection unit 422 selects line data corresponding to the set receiving antenna. For example, the selection unit 422 selects line data of the receiving antenna selected by the selection unit 422 from among the pieces of line data of all of the receiving antennas.

The data collection unit 423 refers to the storage unit 46 or the storage unit 36 of each of the receiving antennas, acquires the line data selected by the selection unit 422, and collects a plurality of pieces of line data that constitute image data. The data collection unit 423 collects the line data of the selected (sorted) receiving antenna, for each of line numbers.

The operating unit 43 is an input device that is used when a user inputs various kinds of setting information or instruction information to the receiving apparatus 4. The operating unit 43 is configured with, for example, a switch, a button, or the like that is arranged on an operation panel of the receiving apparatus 4.

The output unit 44 displays an image, outputs sound or light, or generates vibration. The output unit 44 displays a notification image corresponding to an interference level, or generates sound, light, or vibration. The output unit 44 is configured with at least one of a display, such as a liquid crystal display or an organic electro luminescence (EL) display, a speaker, a light source, and a vibration generator, such as a vibration motor.

The data transmitting/receiving unit 45, when being connected to the processing apparatus 5 in a communicable manner, transmits the image data and the related information that are stored in the storage unit 46 to the processing apparatus 5. The data transmitting/receiving unit 45 is configured with a communication interface, such as a universal serial bus (USB) or a local area network (LAN).

The storage unit 46 stores therein a program for causing the receiving apparatus 4 to operate and implement various functions, image data acquired by the capsule endoscope 2, a threshold for a determination process, and the like. The storage unit 46 is configured with a RAM, a ROM, or the like.

FIG. 4 is a diagram for explaining a configuration of data stored in the storage unit according to the first embodiment of the present disclosure. In the first embodiment, the storage unit 46 stores therein, for each of the receiving antennas, strength data D_0 related to the RSSI of the receiving antenna and pieces of line data D_1 to D_N related to a plurality of lines that constitute an image. Each piece of the line data D_1 to D_N includes information on an image (Image data) and information on the number of bit errors in the line data.

The control unit 47 controls each of the structural units of the receiving apparatus 4. The control unit 47 is configured with a general-purpose processor, such as a CPU, or a dedicated processor, such as various arithmetic circuits including an ASIC or the like, that implements specific functions.

The power supply unit 48 supplies electric power to each of the units of the receiving apparatus 4. The power supply unit 48 is configured with, for example, a battery including an electric battery or the like.

The receiving apparatus 4 as described above is attached to and carried with the subject H while the capsule endoscope 2 is performing imaging, in particular, after the capsule endoscope 2 is swallowed by the subject H and until the capsule endoscope 2 is excreted through digestive tracts. During this period, the receiving apparatus 4 stores image data that is received via the receiving antenna unit 3 in the storage unit 46.

After the capsule endoscope 2 completes imaging, the receiving apparatus 4 is detached from the subject H and set to the cradle 5a (see FIG. 1) that is connected to the processing apparatus 5. Accordingly, the receiving apparatus 4 is connected to the processing apparatus 5 in a communicable manner, and transfers (downloads) the image data and the related information stored in the storage unit 46 to the processing apparatus 5.

The processing apparatus 5 is configured with, for example, a workstation including the display apparatus 6, such as a liquid crystal display. The processing apparatus 5 includes a data transmitting/receiving unit 51, an image processing unit 52, a control unit 53, a display control unit 54, an input unit 55, and a storage unit 56.

The data transmitting/receiving unit 51 is connected to the receiving apparatus 4 via the cradle 5a, and transmits and received data to and from the receiving apparatus 4. The data transmitting/receiving unit 51 is configured with a communication interface, such as a USB or a LAN.

The image processing unit 52 reads a predetermined program stored in a storage unit 58 (to be described later) and performs predetermined image processing for generating an image corresponding to image data that is input from the data transmitting/receiving unit 51 or image data that is stored in the storage unit 58. The image processing unit 52 is implemented by a general-purpose processor, such as a CPU, or a dedicated processor, such as various arithmetic circuits including an ASIC or the like, that implements specific functions.

The control unit 53 reads various programs stored in the storage unit 56, transfers an instruction or data to each of the units included in the processing apparatus 5 on the basis of a signal that is input from the input unit 55 or image data that is input from the data transmitting/receiving unit 51, and comprehensively controls entire operation of the processing apparatus 5. The control unit 53 is implemented by a general-purpose processor, such as a CPU, or a dedicated processor, such as various arithmetic circuits including an ASIC or the like, that implements specific functions.

The display control unit 54 performs predetermined processing, such as data decimation corresponding to an image display range of the display apparatus 6 or gradation processing, on the image generated by the image processing unit 52, and displays and outputs the obtained image together with display target information, such as a final score, on the display apparatus 6. The display control unit 54 is implemented by a general-purpose processor, such as a CPU, or a dedicated processor, such as various arithmetic circuits including an ASIC or the like, that implements specific functions.

The input unit 55 receives input of information or a command corresponding to operation performed by a user. The input unit 55 is implemented by, for example, an input device, such as a keyboard, a mouse, a touch panel, or various switches.

The storage unit 56 stores therein a program for causing the processing apparatus 5 to operate and implement various functions, various kinds of information used during execution of the program, the image data and the related information acquired from the receiving apparatus 4, an endoscopic image generated by the image processing unit 52, and the like. The storage unit 56 is implemented by a semiconductor memory, such as a flash memory, a RAM, or a ROM, a recording medium, such as a hard disk drive (HDD), a magneto-optical disk (MO), a compact disc-recordable (CD-R), or a digital versatile disk-recordable (DVD-R), a driving apparatus that drives the recording medium, or the like.

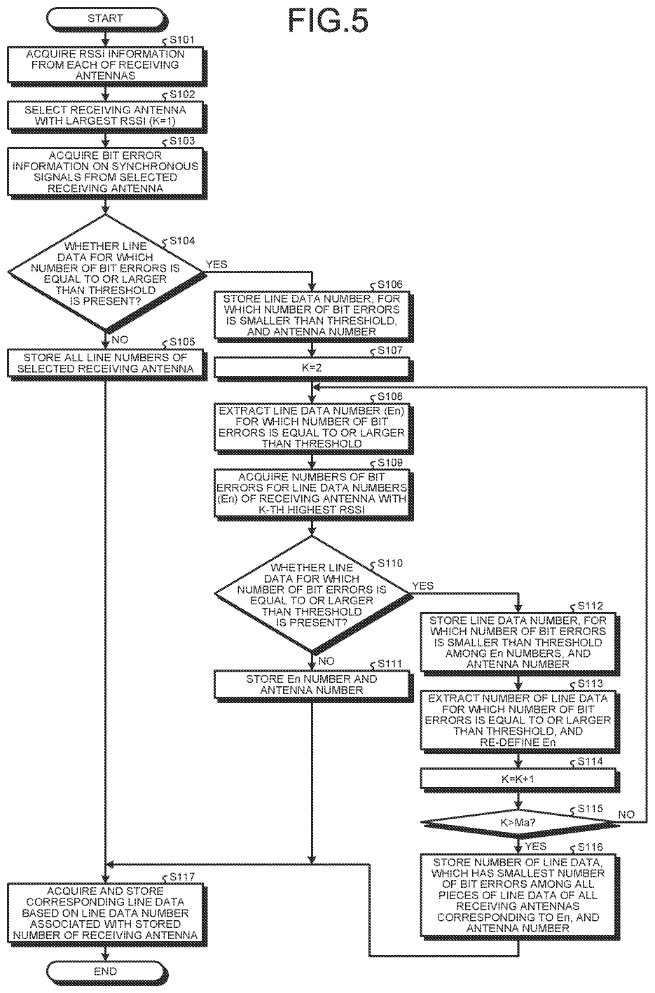

An image data acquisition process performed by the capsule endoscope system 1 will be described below. FIG. 5 is a flowchart illustrating the image data acquisition process performed by the capsule endoscope system according to the first embodiment of the present disclosure. The flowchart illustrated in FIG. 5 is one example of acquiring a plurality of pieces of line data as image data of a single frame.

First, at Step S101, the data processing unit 42 acquires information on the RSSI from each of the receiving antennas. The reception strength processing unit 421 determines a magnitude relationship on the basis of the acquired RSSI of each of the receiving antennas. For example, the reception strength processing unit 421 acquires the strength data D_0 (see FIG. 4) of each of the receiving antennas from the storage unit 46, and determines the magnitude relationship among the RSSIs of all of the receiving antennas.

At Step S102 following Step S101, the selection unit 422 selects a receiving antenna with the largest RSSI on the basis of the magnitude relationship determined by the reception strength processing unit 421. The selection unit 422 sets a selection order K of the receiving antenna with the largest RSSI such that K=1, where the selection order K is for selecting the receiving antenna. The selection unit 422 determines the selection orders with respect to the receiving antennas other than the receiving antenna with the largest RSSI, on the basis of a condition that is preset. For example, the selection unit 422 assigns numbers to the receiving antennas in descending order of the RSSIs (in this example, K=2, 3, . . . , 8). Hereinafter, a maximum value of K (the number of the receiving antennas) is denoted by Ma. In the first embodiment, Ma=8.

At Step S103, the selection unit 422 acquires the bit error information on the selected receiving antenna. The selection unit 422 acquires the number of bit errors of each piece of line data of the selected receiving antenna.

At Step S104, the selection unit 422 determines whether a piece of line data for which the number of bit errors is equal to or larger than a predetermined threshold is present. If the selection unit 422 determines that a piece of line data for which the number of bit errors is equal to or larger than the threshold is present (Step S104: Yes), the process goes to Step S106. In contrast, if the selection unit 422 determines that a piece of line data for which the number of bit errors is equal to or larger than the threshold is not present (Step S104: No), the process goes to Step S105. The threshold is set on the basis of the number of bit errors corresponding to allowable image definition. The threshold is stored in the storage unit 46 in advance. In the first embodiment, a predetermined criterion (first criterion) is that the number of bit errors is smaller than the threshold.

At Step S105, the selection unit 422 associates the receiving antenna with the largest RSSI, with numbers of pieces of line data to be collected, and stores the associated information in the storage unit 46. At Step S105, pieces of line data acquired by the same receiving antenna constitute image data of a single frame. After completion of the association by the selection unit 422, the data processing unit 42 goes to Step S117.

Further, at Step S106, the selection unit 422 selects, as a collection target that is line data to be collected, a piece of line data for which the number of bit errors is smaller than the threshold. The data collection unit 423 stores the line data selected by the selection unit 422 in the storage unit 46 in association with the number of the receiving antenna. In this example, the selected line data and the number of the receiving antenna selected at Step S102 are associated with each other.

At Step S107, the selection unit 422 sets the selection order K of the receiving antenna such that K=2.

At Step S108, the selection unit 422 extracts a number (hereinafter, may be simply referred to as a line number) of line data for which the number of bit errors is determined as being equal to or larger than the threshold at Step S104. The selection unit 422 assigns En to the extracted line number. For example, the selection unit 422 assigns En in ascending order of the line numbers such that En=1, 2, 3, . . . Q (Q is the number of extractions).

At Step S109, the selection unit 422 acquires information on the numbers of bit errors for all pieces of line data to which the line numbers En are assigned with respect to the K-th receiving antenna.

At Step S110, the selection unit 422 determines whether a piece of line data for which the acquired number of bit errors is equal to or larger than a predetermined threshold is present. If the selection unit 422 determines that a piece of line data for which the number of bit errors is equal to or larger than the threshold is present (Step S110: Yes), the process goes to Step S112. In contrast, if the selection unit 422 determines that a piece of line data for which the number of bit errors is equal to or larger than the threshold is not present (Step S110: No), the process goes to Step S111.

At Step S111, the data collection unit 423 acquires En-th line data that is acquired by the K-th receiving antenna. The data collection unit 423 stores the acquired En-th line data as image data of the subject frame in the storage unit 46.

Further, at Step S112, the selection unit 422 selects, as a collection target that is line data to be collected, a piece of line data for which the number of bit errors is smaller than the threshold. The data collection unit 423 stores the line data selected by the selection unit 422 in the storage unit 46 in association with the number of the receiving antenna. In this example, a piece of line data for which the number of bit errors is smaller than the threshold among the pieces of line data assigned with the numbers En with respect to the K-th receiving antenna is associated with the number of the receiving antenna.

At Step S113, the selection unit 422 extracts the line number for which the number of bit errors is determined as being equal to or larger than the threshold at Step S110, and re-defines En. The selection unit 422 re-assigns En to the extracted line number. For example, the selection unit 422 assigns En in ascending order of the line numbers such that En=1, 2, 3, . . . Z (Z is the number of extractions).

At Step S114, the selection unit 422 increments a value of the selection order K of the receiving antenna by one.

At Step S115, the selection unit 422 determines whether the set selection order K is smaller than the maximum value Ma that is allowable for K. If the selection order K is smaller than the maximum value Ma (Step S115: No), the selection unit 422 returns to Step S108 and the above-described processes are repeated for the receiving antenna corresponding to the updated selection order K. In contrast, if the selection order K is equal to the maximum value Ma (Step S115: Yes), the selection unit 422 goes to Step S116.

At Step S116, the selection unit 422 associates line data of the receiving antenna having the smallest number of bit errors, with all pieces of line data assigned with the numbers En, and stores the associated information in the storage unit 46. At this time, if a plurality of receiving antennas having the smallest numbers of bit errors are present, the selection unit 422 associates the receiving antenna with the largest RSSI. At Step 3116, each piece of line data is associated with the receiving antenna corresponding to the selection order K or the receiving antenna with the largest RSSI. After completion of the association by the selection unit 422, the data processing unit 42 goes to Step S117.

At Step S117, the data collection unit 423 collects line data of the receiving antenna associated with each of the line numbers. The data collection unit 423 collects line data that is acquired by the associated receiving antenna, for each of the line numbers.

Through the process as described above, image data of a single frame that is formed of pieces of line data with the line numbers selected from among all of the receiving antennas is generated. The receiving apparatus 4, by repetition of the process as described above, acquires image data of a plurality of frames captured by the capsule endoscope 2.

In the first embodiment as described above, image data of a single frame is generated by pieces of line data each having a high reception strength and the small number of bit errors, on the basis of line data received by each of the receiving antennas. According to the first embodiment, it is possible to obtain image data in which noise is suppressed.

### Second Embodiment

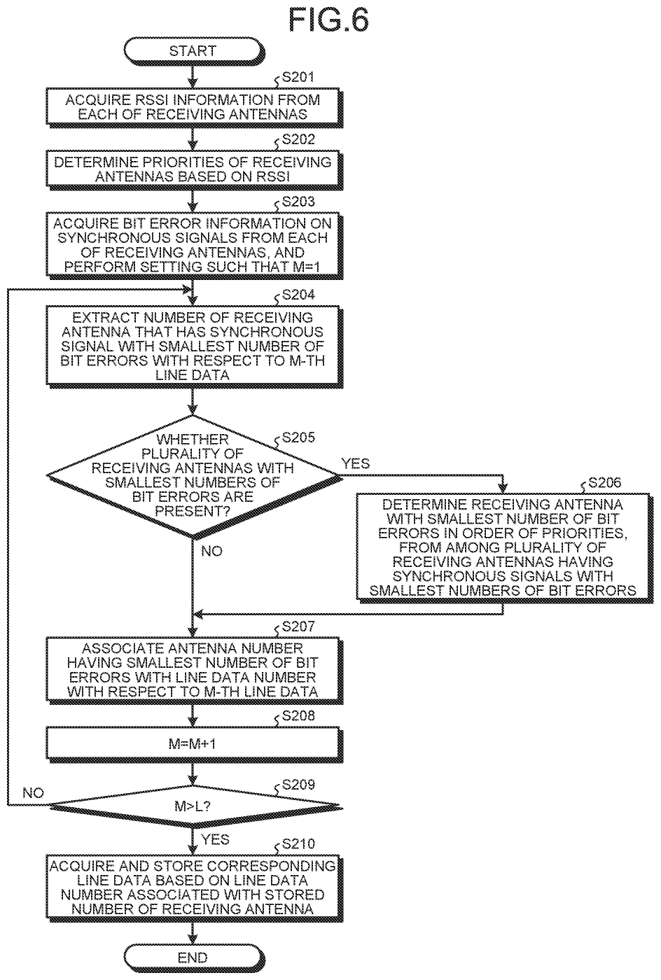

A second embodiment of the present disclosure will be described below. FIG. 6 is a flowchart illustrating an image data acquisition process performed by a capsule endoscope system according to the second embodiment of the present disclosure. The capsule endoscope system according to the second embodiment has the same configuration as that of the first embodiment. A process different from the first embodiment will be described below with reference to FIG. 6.

First, at Step S201, the data processing unit 42 acquires information on the RSSI from each of the receiving antennas. Subsequently, the reception strength processing unit 421 determines priorities on the basis of the acquired RSSI of each of the receiving antennas (Step S202). In the second embodiment, the priorities are determined in descending order of the RSSIs.

At Step S203 following Step S202, the selection unit 422 acquires the number of bit errors of each piece of line data of each of the receiving antennas. A line number M is set such that M=1.

At Step S204, the selection unit 422 extracts a receiving antenna that has line data with the smallest number of bit errors (best line data) among the receiving antennas with respect to the M-th line data.

At Step S205 following Step S204, the selection unit 422 determines whether a plurality of receiving antennas with the smallest numbers of bit errors are present with respect to each of line numbers of the collection target. If the selection unit 422 determines that a plurality of receiving antennas with the smallest number of bit errors are present (Step S205: Yes), the process goes to Step S206. If the selection unit 422 determines that only a single receiving antenna with the smallest number of bit errors is present (Step S205: No), the process goes to Step S207. A first condition is that a plurality of antennas with the smallest numbers of bit errors are not present at Step S205.

At Step S206, the selection unit 422 determines an antenna number with the smallest number of bit errors in order of priorities based on the RSSIs, from among the plurality of antennas with the smallest numbers of bit errors. Meanwhile, a second condition is that the priority is the highest when the plurality of receiving antennas with the smallest numbers of bit errors are present at Step S206.

At Step S207, the selection unit 422 associates, with respect to each piece of line data, the line data number with an antenna number that has the synchronous signal for which the number of bit errors is the smallest. The selection unit 422 stores the associated information in the storage unit 46. After completion of the association, the control unit 47 goes to Step S208.

At Step S208, the selection unit 422 increments a value of the line number M by one.

At Step S209 following Step S208, if the line number M is larger than a maximum value L (Step S209: Yes), the selection unit 422 goes to Step S210. In contrast, if the line number M is equal to or smaller than the maximum value L (Step S209: No), the selection unit 422 goes to Step S204.

The selection unit 422 repeats the processes from Step S204 to Step S209 until all of the line data numbers are associated with the antenna numbers.

At Step S210, the data collection unit 423 collects line data of the receiving antenna associated with each of the line numbers. The data collection unit 423 collects line data that is acquired by the associated receiving antenna, for each of the line numbers.

Through the process as described above, image data of a single frame that is formed of pieces of line data with the line numbers selected from among all of the receiving antennas is generated.

In the second embodiment as described above, if the receiving antennas that have the same numbers of bit errors are present with respect to line data with the same number, the receiving antenna with the higher priority that is determined based on the RSSI is selected. In the second embodiment, image data of a single frame is generated by pieces of line data each having a high reception strength and the small number of bit errors, on the basis of the line data received by each of the receiving antennas.

According to the second embodiment, it is possible to obtain image data in which noise is suppressed.

### Third Embodiment

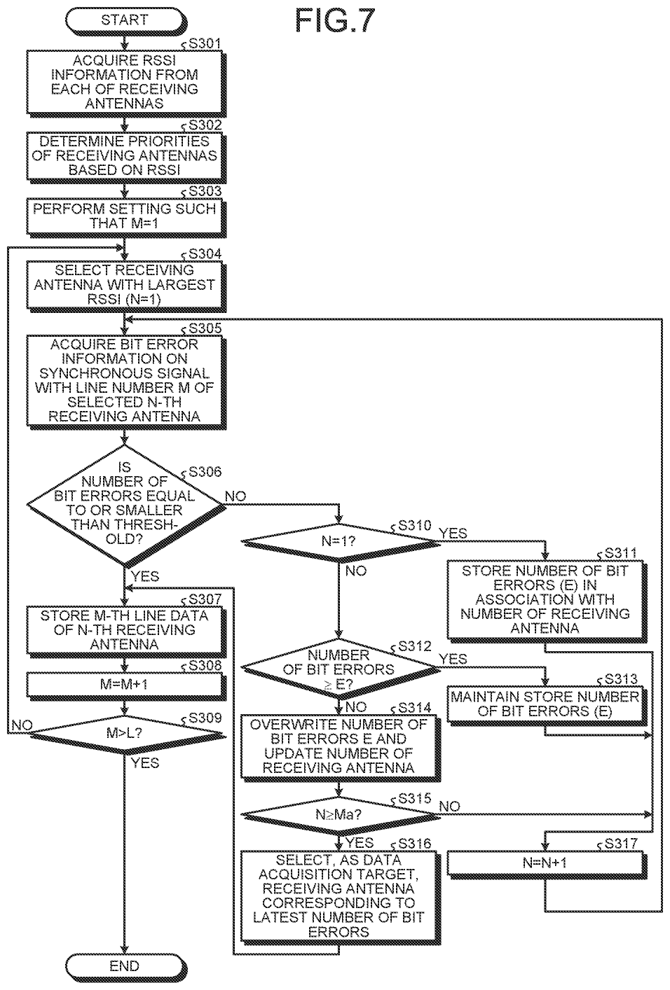

A third embodiment of the present disclosure will be described below. FIG. 7 is a flowchart illustrating an image data acquisition process performed by a capsule endoscope system according to the third embodiment of the present disclosure. The capsule endoscope system according to the third embodiment has the same configuration as that of the first embodiment. A process different from the first embodiment will be described below with reference to FIG. 7.

First, at Step S301, the data processing unit 42 acquires information on the RSSI from each of the receiving antennas. Subsequently, the reception strength processing unit 421 determines priorities on the basis of the acquired RSSI of each of the receiving antennas (Step S302). In the third embodiment, priorities N are determined in descending order of the RSSIs (N=1, 2, 3, . . . , Ma). The selection unit 422 assigns numbers to all of the receiving antennas in descending order of the RSSIs, for example. The priorities in the third embodiment are the same as the selection orders as described above.

At Step S303 following Step S302, the selection unit 422 sets an association target line number M such that M=1. In the third embodiment, a maximum value of the line number is denoted by L.

At Step S304 following Step S303, the selection unit 422 selects a receiving antenna with the largest RSSI on the basis of the magnitude relationship determined by the reception strength processing unit 421. The selection unit 422 sets the priority N of the receiving antenna with the largest RSSI such that N=1.

At Step S305, the selection unit 422 acquires the bit error information on the M-th line number of the receiving antenna with the priority N.

At Step S306, the selection unit 422 determines whether the acquired number of bit errors is equal to or smaller than a predetermined threshold. If the selection unit 422 determines that the number of bit errors is larger than the threshold (Step S306: No), the process goes to Step S310. In contrast, if the selection unit 422 determines that the number of bit errors is equal to or smaller than the threshold (Step S306: Yes), the process goes to Step S307. Meanwhile, a first condition is that the number of bit errors is equal to or smaller than the threshold at Step S306.

At Step S307, the data collection unit 423 stores line data with the line number M of the N-th receiving antenna in the storage unit 46. After the selection unit 422 completes the association, the data processing unit 42 goes to Step S308.

At Step S308 following Step S307, the selection unit 422 increments a value of the line number M by one.

At Step S309 following Step S308, the selection unit 422 determines whether the set line number M is equal to or smaller than the maximum value L that is allowable for M. If the line number M is larger than the maximum value L (Step S309: Yes), the selection unit 422 goes to Step S318. In contrast, if the line number M is equal to or smaller than the maximum value L (Step S309: No), the selection unit 422 returns to Step S304.

Further, at Step S310, the selection unit 422 determines whether the priority N of the set receiving antenna is set such that N=1. If the selection unit 422 determines that N is not set such that N=1 (Step S310: No), the process goes to Step S312. In contrast, if the selection unit 422 determines that N is set such that N=1 (Step S310: Yes), the process goes to Step S311.

At Step S311, the selection unit 422 associates the number of bit errors E of the line number M of the receiving antenna for which N=1 with the number of the receiving antenna, and stores the associated information in the storage unit 46.

Further, at Step S312, the selection unit 422 acquires the bit error information on the line number M of the N-th receiving antenna, and determines whether the acquired number of bit errors is equal to or larger than the stored number of bit errors E. If the selection unit 422 determines that the acquired number of bit errors is smaller than the stored number of bit errors E (Step S312: No), the process goes to Step S314. In contrast, if the selection unit 422 determines that the acquired number of bit errors is equal to or larger than the stored number of bit errors E (Step S312: Yes), the process goes to Step S313.

At Step S313, the selection unit 422 maintains the stored number of bit errors E.

Further, at Step S314, the selection unit 422 updates the number of bit errors E with the number of bit errors acquired at Step S311.

At Step S315 following Step S314, the selection unit 422 determines whether the set priority N is equal to or larger than the maximum value Ma that is allowable for N. If the selection unit 422 determines that the priority N is smaller than the maximum value Ma (Step S315: No), the process goes to Step S317. In contrast, if the selection unit 422 determines that the priority N is equal to the maximum value Ma (Step S315: Yes), the process goes to Step S316.

At Step S316, the selection unit 422 selects, as an acquisition target, a piece of line data with the line number M of the receiving antenna corresponding to the number of bit errors E. After selection of the receiving antenna, the data processing unit 42 goes to Step S307. Meanwhile, a second condition is that if the first condition is not met, the number of bit errors is the smallest among all of the receiving antennas at Steps S307 to S316.

At Step S317, the selection unit 422 sets the priority N such that N=N+1. After setting the priority, the data processing unit 42 goes to Step S305 and repeats the process as described above.

Through the process as described above, image data of a single frame that is formed of pieces of line data with the line numbers selected from among all of the receiving antennas is generated.

In the third embodiment as described above, line data of the receiving antenna with the largest RSSI is used as a base, and line data of a different receiving antenna is selected as line data with the large number of bit errors. In the third embodiment, image data of a single frame is generated by pieces of line data each having the small number of bit errors, on the basis of the line data received by each of the receiving antennas. According to the third embodiment, it is possible to obtain image data in which noise is suppressed.

### Fourth Embodiment

A fourth embodiment of the present disclosure will be described below. In the first to the third embodiments as described above, acquisition target line data is selected by using the RSSI of each of the receiving antennas and the bit error information on the synchronous signals of a plurality of pieces of line data included in the image data. In contrast, in the fourth embodiment, acquisition target line data is selected by using only the RSSI that is measured every time each piece of line data included in image data is received. A capsule endoscope system according to the fourth embodiment is configured by removing the error counting unit 342 from the configuration of the capsule endoscope 1 according to the first embodiment. Other configurations of the capsule endoscope system according to the fourth embodiment are the same as those of the first embodiment.

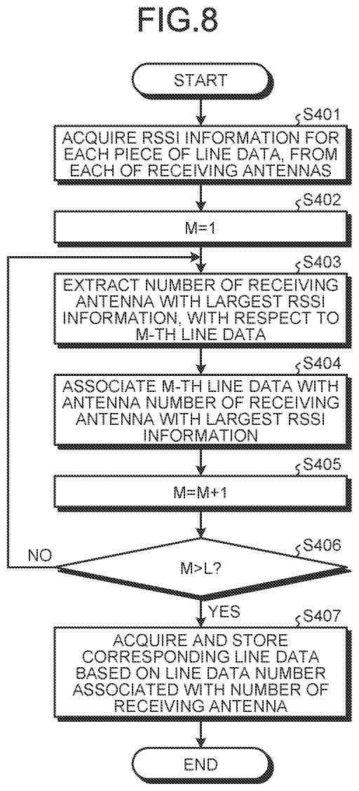

FIG. 8 is a flowchart illustrating an image data acquisition process performed by the capsule endoscope system according to the fourth embodiment of the present disclosure. A process according to the fourth embodiment will be described below with reference to FIG. 8.

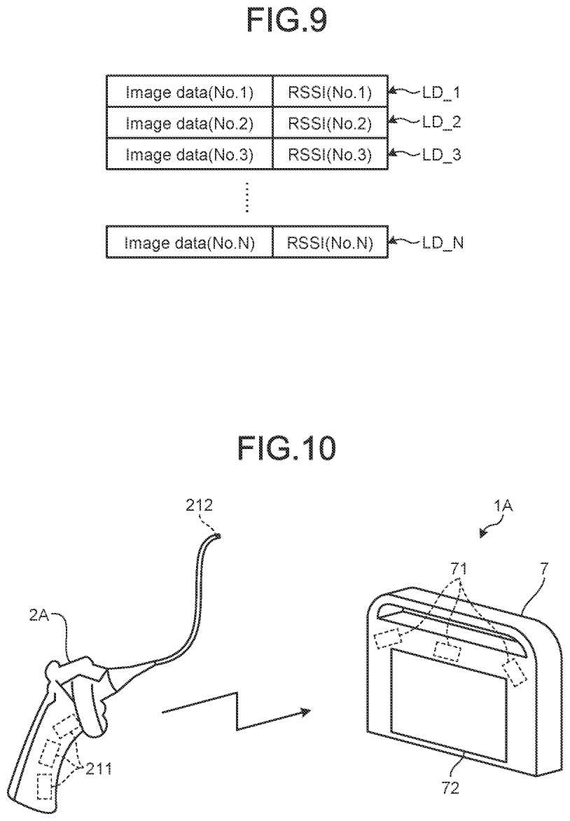

First, at Step S401, the data processing unit 42 acquires information on the RSSI for each piece of line data, from each of the receiving antennas. FIG. 9 is a diagram for explaining a configuration of line data used in the fourth embodiment of the present disclosure. In the fourth embodiment, the data processing unit 42 acquires each piece of line data (LD_1 to LD_N) that includes information on an image (Image data) and RSSI information (RSSI), and extracts the RSSI information from each piece of the acquired line data. In the fourth embodiment, the information illustrated in FIG. 9 is stored in the storage unit 46.

At Step S402, the selection unit 422 acquires the number of bit errors of line data of each of the receiving antennas. The line number M is set such that M=1.

At Step S403 following Step S402, the selection unit 422 extracts the antenna number of the receiving antenna having line data with the largest RSSI with respect to the M-th line data.

At Step S404 following Step S403, the selection unit 422 associates the M-th line data with the antenna number of the receiving antenna with the largest RSSI. The selection unit 422 stores the associated information in the storage unit 46. After the association, the control unit 47 goes to Step S405.

At Step S405 following Step S404, the selection unit 422 increments a value of the line number M by one.

At Step S406 following Step S405, if the line number M is larger than the maximum value L (Step S406: Yes), the selection unit 422 goes to Step S407. In contrast, if the line number M is not larger than the maximum value L (Step S406: No), the selection unit 422 goes to Step S403.

The selection unit 422 repeats the processes from Step S403 to Step S406 until all of the line data numbers are associated with the antenna numbers.

At Step S407, the data collection unit 423 collects line data of the receiving antenna associated with each of the line numbers. The data collection unit 423 collects line data that is acquired by the associated receiving antenna, for each of the line numbers.

Through the process as described above, image data of a single frame that is formed of pieces of line data with the line numbers selected from among all of the receiving antennas is generated.

In the fourth embodiment as described above, the receiving antenna from which image data is to be acquired is selected based on the RSSI. In the fourth embodiment, image data of a single frame is generated by pieces of line data each having a high reception strength, on the basis of the line data received by each of the receiving antennas. According to the fourth embodiment, it is possible to obtain image data in which noise is suppressed.

Meanwhile, if a difference between the RSSI of the line data with the line number M−1 and the RSSI of the line data with the line number M is equal to or smaller than a reference value, it may be possible to adopt the receiving antenna of the line data that is selected as an acquisition target for the line number M−1, as the receiving antenna of the line data to be selected as an acquisition target for the line number M.

Specifically, if a difference between the RSSI of a receiving antenna N of the line data that is selected as an acquisition target for the line number M−1 and the RSSI of the same receiving antenna for the line number M is equal to or smaller than a reference value, line data that is assigned with the line number M and that is received by the same receiving antenna N is selected as an acquisition target.

In the fourth embodiment as described above, image data of a single frame is generated by selecting line data of the receiving antenna with the largest RSSI, for each piece of line data. According to the fourth embodiment, it is possible to obtain image data in which noise is suppressed.

While the embodiments of the present disclosure have been explained above, the present disclosure is not limited to the embodiments and the modifications as described above. The present disclosure is not limited to the embodiments and the modifications as described above, and includes various other embodiments within a scope not departing from the technical idea described in the appended claims. Further, the configurations of the embodiments and the modifications may be combined appropriately.

Meanwhile, in the first to the fourth embodiments as described above, the capsule endoscope 2 has been described as an example of the medical apparatus, but the present disclosure is not limited to this example. For example, a medical apparatus, such as an implant medical device or a catheter, that is introduced into a subject, that acquires pH information, and that outputs the acquired information as a wireless signal may be adopted. Furthermore, a medical apparatus that outputs, as a wireless signal, an image signal that is captured by an endoscope having a different system from a capsule type, e.g., an endoscope that performs wireless communication with a processing apparatus, may be adopted.

### Fifth Embodiment

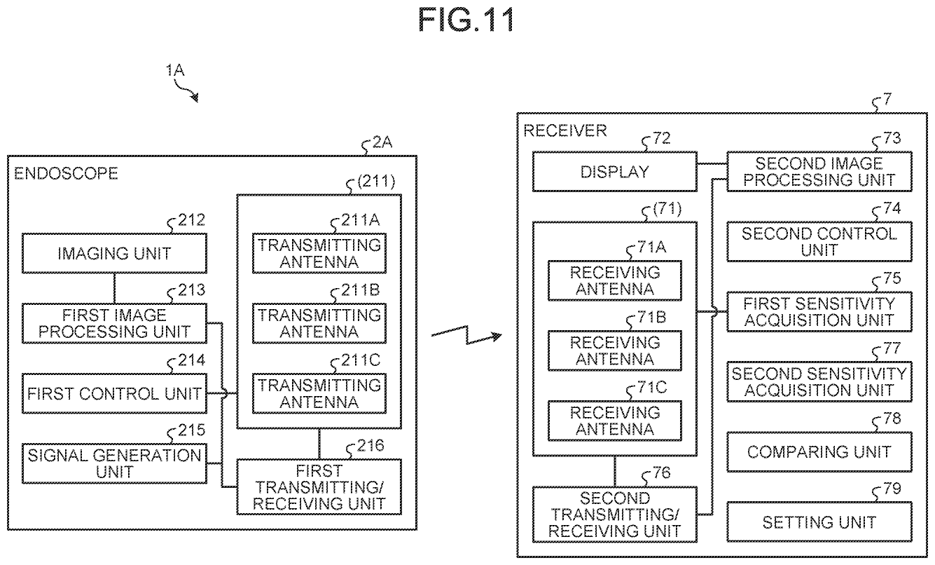

A fifth embodiment of the present disclosure will be described below. FIG. 10 is a schematic diagram illustrating a schematic configuration of an endoscope system according to the fifth embodiment of the present disclosure. FIG. 11 is a block diagram illustrating a schematic configuration of the endoscope system according to the fifth embodiment of the present disclosure. In the first to the fourth embodiments as described above, the capsule endoscope system including the capsule endoscope 2 has been described, but in the fifth embodiment, an example of an endoscope system that includes a wireless endoscope 2A capable of performing wireless communication and a receiver 7 capable of performing communication with the wireless endoscope 2A will be described.

The wireless endoscope 2A (hereinafter, may be simply referred to as the “endoscope 2A”) includes, for example, an imaging unit 212 at a distal end portion of a thin and elongated insertion portion thereof. The receiver 7 that is a portable monitor includes receiving antennas 71 and a display 72 that displays an endoscopic image. Transmitting antennas 211 of the endoscope 2A and the receiving antennas 71 of the receiver 7 perform wireless communication using a 5 GHz band or a 60 GHz band.

The endoscope 2A includes the transmitting antennas 211 (transmitting antennas 211A to 211C), the imaging unit 212, a first image processing unit 213, a first control unit 214, a signal generation unit 215, and a first transmitting/receiving unit 216.

The imaging unit 212 includes an image sensor and acquires an endoscopic image. The first image processing unit 213 performs predetermined image processing, such as image compression processing, on the endoscopic image, and outputs an image signal. The first control unit 214 controls the entire operation of the endoscope 2A and controls wireless transmission and reception, for example, performs switching control on the transmitting antennas 211 as will be described later. The signal generation unit 215 generates a signal for acquiring a second sensitivity to be described later. The first transmitting/receiving unit 216 transmits and receives a signal by using the transmitting antennas 211. A wireless signal transmitted and received by the first transmitting/receiving unit 216 includes a main signal including the image signal and includes a signal (sub signal) that is generated by the signal generation unit 215 and that is transmitted at a carrier frequency different from the main signal.

The receiver 7 includes the receiving antennas 71 (receiving antennas 71A to 71C), the display 72, a second image processing unit 73, a second control unit 74, a first sensitivity acquisition unit 75, a second transmitting/receiving unit 76, a second sensitivity acquisition unit 77, a comparing unit 78, and a setting unit 79. The receiver 7 includes a storage unit in the receiver 7 or the second control unit 74.

The second transmitting/receiving unit 76 transmits and receives a signal by using the receiving antennas 71. The second image processing unit 73 performs predetermined image processing on a received image signal, and displays an image on the display 72 under the control of the second control unit 74. The second control unit 74 controls the entire receiver 7 and controls wireless transmission and reception, for example, performs switching control on the receiving antennas 71. The first sensitivity acquisition unit 75, the second sensitivity acquisition unit 77, the comparing unit 78, and the setting unit 79 acquire data for performing the switching control on antenna pairs that are sets of the transmitting antennas 211 and the receiving antennas 71. Meanwhile, as for the antenna pairs, sets of the receiving antennas and the transmitting antennas may be preset or setting of the pairs may be changed appropriately.

The first sensitivity acquisition unit 75 acquires a first communication sensitivity that is a communication sensitivity (that is, reception strength or RSSI) of a first antenna pair that is formed of a first transmitting antenna and a first receiving antenna and that is a set of a transmitting antenna and a receiving antenna for transmitting and receiving an image signal that is a main signal having largest transmission quantity among wireless signals. The second sensitivity acquisition unit 77 acquires, by using a sub signal, second communication sensitivities of a plurality of second antenna pairs each being formed of any of the transmitting antennas and any of the receiving antennas that do not transmit and receive the main signal. Further, if a communication sensitivity between a second transmitting antenna and a second receiving antenna, which is the highest among the plurality second communication sensitivities, is higher than the first communication sensitivity, the setting unit 79 sets the antenna pair formed of the second transmitting antenna and the second receiving antenna as the first antenna pair that transmits and receives the main signal.

In the fifth embodiment, similarly to the image data acquisition process of the first embodiment, an image data acquisition process is performed (see FIG. 5). Specifically, the second control unit 74 acquires the RSSI information received by each of the receiving antennas (the receiving antennas 71A to 71C) of the receiver 7, selects a receiving antenna with the largest RSSI, adopts the selected receiving antenna as the first receiving antenna, and acquires the bit error information on the synchronous signals of the receiving antenna. At this time, if the numbers of bit errors of the selected receiving antenna are smaller than a threshold, the second control unit 74 stores all pieces of line data acquired by the receiving antenna in the storage unit. In contrast, if the number of bit errors of the selected receiving antenna is equal to or larger than the threshold, the second control unit 74 stores, in the storage unit, line data of a receiving antenna that is one of the other receiving antennas (the second receiving antennas), that has the smaller number of bit errors than the threshold among the receiving antennas having smaller RSSIs than the selected antenna, and that has the largest RSSI among the other receiving antennas.

In the fifth embodiment as described above, similarly to the first embodiment, image data of a single frame is generated by pieces of line data each having a high reception strength and the small number of bit errors, on the basis of line data received by each of the receiving antennas. Therefore, it is possible to obtain image data in which noise is suppressed.

Furthermore, an endoscope system 1A according to the fifth embodiment suppresses reduction in the communication sensitivity by transmitting and receiving the image signal by using an antenna pair with the highest communication sensitivity among the plurality of antennas. According to the endoscope system 1A, even if relative positions of the endoscope 2A and the receiver 7 are changed or an object that disturbs radio wave transmission is provided between the endoscope 2A and the receiver, it is possible to transmit a high quality endoscopic image.

Meanwhile, in the first to the third embodiments as described above, the example has been described in which the error counting unit 342 counts the number of bit errors in the synchronous signal as pattern data, but it may be possible to count the number of bits of a normally acquired synchronous signal. In this case, line data for which the counted value is larger than a threshold is selected as an acquisition target. In this case, a second criterion is that the counted value is larger than the threshold. Further, the error counting unit 342 may be configured to count the number of fixed patterns or the number of pieces of block data included in each piece of line data, instead of the synchronous signal.

Furthermore, in the first to the fifth embodiments as described above, the example has been described in which the data processing unit 42 performs processing for each piece of line data, but the collection target is not limited to data of each line, but a plurality of pieces of line data may be collected as a single set. For example, three consecutive pieces of line data may be grouped into a single set, the error counting unit 342 may count a representative count value of each of the sets, and the data collection unit 423 may collect line data for each of the sets. In the present embodiment, each piece of partial image data that constitutes image data of a single frame may be adopted as a single piece of line data or may be adopted as a single set of line data.

Moreover, in the first to the fifth embodiments as described above, the example has been described in which the process of counting the number of bit errors or the like is performed for all of the receiving antennas, but it may be possible to perform the process for a part of the receiving antennas. For example, it may be possible to perform the process on only the receiving antennas for which the RSSIs are equal to or larger than a predetermined value. In other words, it may be possible to generate groups of receiving antennas based on the RSSIs, and perform the process on only a group of the receiving antennas having large RSSIs.

Furthermore, an execution program implemented by each of the structural units of the capsule endoscope systems 1 and 1A in the embodiments may be provided by being recorded in a computer readable recording medium, such as a compact disc-read only memory (CD-ROM), a flexible disk (FD), a CD-R, or a digital versatile disk (DVD), in an installable or an executable file format, or may be provided by being stored in a computer connected to a network, such as the Internet, and by being downloaded via the network. Moreover, the execution program may be provided or distributed via a network, such as the Internet.

As described above, the receiving system according to the present disclosure is useful for acquiring image data in which noise is suppressed.

According to the present disclosure, it is possible to acquire image data in which noise is suppressed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A processing apparatus comprising:
a processor configured to:
acquire, for each of receiving antennas receiving wireless signals that are transmitted from a medical apparatus configured to capture an image of an inside of a subject, a plurality of pieces of line data that constitute first image data from the wireless signals,
count numbers related to acquisition of synchronous signals included in the plurality of pieces of line data, and
collect a plurality of pieces of line data that constitute second image data from the plurality of pieces of line data that are acquired by the receiving antennas and that constitute a plurality of pieces of the first image data, based on the numbers related to acquisition.

2. The processing apparatus according to claim 1, wherein
the processor is configured to
measure, for each of the receiving antennas, reception strengths of the wireless signals received by the receiving antennas, and
collect the plurality of pieces of line data that constitute the second image data from the plurality of pieces of line data that are acquired by the receiving antennas and that constitute the plurality of pieces of first image data, based on the reception strengths and the numbers related to acquisition.

3. The processing apparatus according to claim 2, wherein
when collecting first line data that constitute a part of the second image data, the processor is configured to
collect a piece of line data that is acquired by a receiving antenna with a lowest reception strength among the pieces of line data for which the counted numbers meet a predetermined criterion, the and
adopt the piece of collected line data as the first line data.

4. The processing apparatus according to claim 3, wherein the processor is configured to determine whether the counted numbers meet the predetermined criterion in ascending order of the reception strengths.

5. The processing apparatus according to claim 2, wherein the numbers related to acquisition indicate one of numbers of normally received synchronous signals or numbers of errors in the synchronous signals for the respective pieces of line data.

6. The processing apparatus according to claim 5, wherein
with respect to the pieces of line data that are assigned with same numbers and that are received by the respective receiving antennas, the processor is configured to
collect a piece of line data that is acquired from a receiving antenna with a highest reception strength among the pieces of line data for which either the numbers of the normally received synchronous signals or the numbers of errors in the synchronous signals meet a predetermined criterion, and
adopt the piece of collected line data as first line data that constitute a part of the second image data.

7. The processing apparatus according to claim 5, wherein
with respect to the pieces of line data that are assigned with same numbers and that are received by the respective receiving antennas, if a plurality of pieces of line data for which the numbers of the normally received synchronous signals are largest are present or if a plurality of pieces of line data for which the numbers of errors in the synchronous signals are smallest are present among pieces of line data that are assigned with same numbers and that are received by the respective receiving antennas, the processor is configured to
collect a piece of line data that is acquired from a receiving antenna with a highest reception strength among the pieces of line data, and
adopt the piece of collected line data as first line data that constitute a part of the second image data.

8. The processing apparatus according to claim 2, wherein
the processor is configured to
classify the receiving antennas into a plurality of groups, and
collect the plurality of pieces of line data that constitute the second image data based on the reception strengths and the numbers related to acquisition with respect to the receiving antennas that belong to a specific group.

9. The processing apparatus according to claim 1, wherein the processor is configured to collect the plurality of pieces of line data that constitute the second image data, for each piece of the line data.

10. The processing apparatus according to claim 1, wherein the processor is configured to collect the plurality of pieces of line data that constitute the second image by adopting the plurality of pieces of line data as a set.

11. The processing apparatus according to claim 1, wherein the medical apparatus is a capsule endoscope or an endoscope.

12. The processing apparatus according to claim 1, wherein
the medical apparatus includes transmitting antennas including a first transmitting antenna transmitting a wireless signal and a second transmitting antenna transmitting a wireless signal, and
the receiving antennas includes a first receiving antenna and a second receiving antenna.

13. The processing apparatus according to claim 12, wherein the processor is configured to
acquire a first communication sensitivity of an antenna pair being formed of the first transmitting antenna and the first receiving antenna and transmitting and receiving a main signal in the wireless signals,
acquire a second communication sensitivity of each of antenna pairs each being formed of any of the transmitting antennas and any of the receiving antennas and transmitting and receiving a sub signal in the wireless signals, and
if a communication sensitivity between the second transmitting antenna and the second receiving antenna, the communication sensitivity being highest among a plurality of second communication sensitivities, is higher than the first communication sensitivity, set an antenna pair formed of the second transmitting antenna and the second receiving antenna as an antenna pair transmitting and receiving the main signal.

14. A processing method implemented by a processor, the processing method comprising:
acquiring, for each of receiving antennas receiving wireless signals that are transmitted from a medical apparatus configured to capture an image of an inside of a subject, a plurality of pieces of line data that constitute first image data from the wireless signals;
counting numbers related to acquisition of synchronous signals included in the plurality of pieces of line data; and
collecting a plurality of pieces of line data that constitute second image data from the plurality of pieces of line data that are acquired by the receiving antennas and that constitute a plurality of pieces of the first image data, based on the numbers related to acquisition.

15. The processing method according to claim 14, further comprising:
measuring, for each of the receiving antennas, reception strengths of the wireless signals received by the receiving antennas; and
collecting the plurality of pieces of line data that constitute the second image data from the plurality of pieces of line data that are acquired by the receiving antennas and that constitute the plurality of pieces of first image data, based on the reception strengths and the numbers related to acquisition.

16. A non-transitory computer readable recording medium having an executable program recorded thereon, the program instructing a processor to execute:
acquiring, for each of receiving antennas receiving wireless signals that are transmitted from a medical apparatus configured to capture an image of an inside of a subject, a plurality of pieces of line data that constitute first image data from the wireless signals;
counting numbers related to acquisition of synchronous signals included in the plurality of pieces of line data; and
collecting a plurality of pieces of line data that constitute second image data from the plurality of pieces of line data that are acquired by the receiving antennas and that constitute a plurality of pieces of the first image data, based on the numbers related to acquisition.

17. The recording medium according to claim 16, further comprising:
measuring, for each of the receiving antennas, reception strengths of the wireless signals received by the receiving antennas; and
collecting the plurality of pieces of line data that constitute the second image data from the plurality of pieces of line data that are acquired by the receiving antennas and that constitute the plurality of pieces of first image data, based on the reception strengths and the numbers related to acquisition.
